(19) United States
(12) Patent Application Publication
(10) Pub. No.: US 2004/0170669 A1
(43) Pub. Date: Sep. 2, 2004

(54) Feed rations and methods of feeding growing ruminants

(76) Inventors: William E. Kunkle, Gainsville, FL (US); Edgar Rodriguez, Barquisimeto (VE); Mercedes Vazquez-Anon, Chesterfield, MO (US); Bryan G. Miller, Warrenton, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/623,881

(22) Filed: Jul. 21, 2003

Related U.S. Application Data
(60) Provisional application No. 60/397,156, filed on Jul. 19, 2002.
(60) Provisional application No. 60/397,957, filed on Jul. 22, 2002.

(51) Int. Cl. A61K 38/00; A61K 31/19

(52) U.S. Cl. 424/442; 514/2; 514/562; 514/557

(57) ABSTRACT

The present invention relates to feed rations, processes for formulating feed rations, and methods for improving weight gain in growing ruminants. The feed rations comprise a forage or other feed ingredient that meets daily nutritional requirements, other than methionine, and exceeds daily maintenance energy requirements of a growing ruminant. The feed rations further comprise an α-amino acid supplement, wherein the α-amino acid supplement is an analog of an α-amino acid.

### CROSS-REFERENCE TO RELATED APPLICATION

This application claims the benefit of U.S. provisional application Serial No. 60/397,957, filed Jul. 22, 2002, and U.S. provisional application Serial No. 60/397,156, filed Jul. 19, 2002, the entire disclosures of which are incorporated herein by reference.

### FIELD OF THE INVENTION

The present invention relates, in general, to ruminant feed rations, processes for formulating feed rations, and methods for feeding growing ruminants. More particularly, the present invention relates to improving weight gain in growing ruminants through providing ruminants a feed ration that meets the ruminant's nutrient requirements, or is deficient in methionine but otherwise meets the ruminant's daily nutritional requirements, and exceeds their maintenance energy requirements, wherein the feed ration further comprises an amino acid supplement, wherein the amino acid supplement is an analog of an α-amino acid.

### BACKGROUND OF INVENTION

Improving nutrition for growing ruminants, such as beef and dairy cattle provides a means of improving production in the cattle industry. This is particularly important during the late summer through winter or even through early spring, such as early April, when forage availability and quality are limiting.

In order to meet the nutritional requirements of ruminants, some ranchers have provided feed supplements such as blackstrap molasses to cow herds as a winter supplement to their forage diets. Sources and levels of protein added to molasses-based liquid supplements fed to growing cattle has been a focus of research. Experiments have evaluated different levels, sources (soybean meal, cottonseed meal, feather meal, blood meal) and combinations of protein in molasses-based supplements for growing steers and heifers fed tropical forages (Kunkle et al., Proc. 5th Annual Florida Ruminant Nutrition Symposium, pp 89-103 (1994), Univ. of Florida, Gainesville, Fla.; Stateler et al., J. Anim. Sci. 73:3078-3084 (1995); Pate et al., J. Anim. Sci. 73:2865-2872 (1995); Brown, J. Anim. Sci. 71:3451-3457 (1993)). The experiments provided ruminants with non-protein nitrogen (NPN) ingredients or rumen degradable intake protein (DIP) that can be utilized by rumen bacteria and then evaluated responses to additional protein from sources that provided rumen undegraded intake protein (UIP). The UIP digested by ruminants is broken down to component amino acids that are absorbed in the small intestine.

Kunkle et al. (1994) found an increase in weight gain from several sources and levels of supplemental protein containing UIP. Kunkle concluded that feeding sources of UIP to growing beef cattle improved their performance and was profitable in selected beef production systems.

While forage-fed growing cattle offered molasses-based supplements improved gains when UIP was added to the supplements, the early research did not identify the optimum level for each UIP source or how to compare sources of UIP.

In ruminants, one or two amino acids have been found to limit performance. Bergen et al. stated that methionine is the first limiting amino acid in microbial protein (Bergen et al., J. Nutr. 92:357-364 (1967)). In lactating dairy cattle fed corn-based diets, Schwab et al. stated that methionine and lysine are the first and second limiting amino acids for milk production (Schwab et al., J. Dairy Sci. 75:3486-3502 (1992)).

Kunkle et al. (1999) conducted experiments wherein steers and heifers were offered Bermuda grass hay ad libitum and molasses slurries containing molasses, urea, corn, and different sources of UIP or methionine. The UIP or methionine sources were selected from blood/feather meal (50:50 ratio), corn gluten meal, and rumen-protected methionine encapsulated in a pH sensitive polymer. A comparison of methionine intake on daily weight gain indicated that the weight gain increased linearly with each gram of UIP or methionine source. All three sources gave similar improvements in weight gain suggesting that methionine was the first limiting amino acid in growing cattle fed Bermuda grass hay supplemented with molasses (Proc. 10th Annual Florida Ruminant Nutrition Symposium, pp 19-29, (1999) Univ. of Florida, Gainesville, Fla.).

Popovici et al. (1983) conducted an experiment on 0 to 6 month old calves wherein the diet of test calves was supplemented with the calcium salt of 2-hydroxy-4-(methylthio)butanoic acid. Test calves had an increased average daily weight gain of 8-10% over control calves consuming a diet that did not contain the calcium salt of 2-hydroxy-4-(methylthio)butanoic acid. Both test and control calves were fed a ration containing forage (2 kg/calf/day hay) and a compound feed (2.6-2.8 kg/calf/day). The compound feed contained ground corn cobs (50%), barley (20%), wheat bran (10%), sunflower scrap (15%), feed chalk (1%), bone flour (1%), salt (2%), and ZOOFORT T1 (1%). (Nutrition Abstracts Review, 53(10), 640 (1983)).

Experiments were also conducted wherein rumen-bypass methionine sources were correlated to weight gain in growing beef cattle. Rumen-bypass methionine included corn gluten meal or Mepron M85®, which is DL-methionine encapsulated in a rumen-protecting polymer, (Degussa Corporation, Ridgefield Park, N.J.). The experimental results indicated that dose-related weight gains were present in cattle fed corn gluten meal. In contrast, dose-related weight gains were not observed in cattle fed MEPRON M85® (Davis, et al., 2000, J. Anim. Sci. 78:116).

The forage based feed rations and supplements of the past have been inconsistent in providing weight gain results in growing ruminants. A feed ration and method that consistently results in improving weight gain in growing ruminants would therefore be beneficial to the productivity of the ruminant industry.

### SUMMARY OF THE INVENTION

Among the various aspects of the present invention, therefore, is the provision of a method for improving the weight gain in a growing ruminant, the provision of a process to formulate a feed ration for a growing ruminant, and the provision of a feed ration for growing ruminants.

Briefly, therefore, the present invention is directed to a method of improving weight gain of a growing ruminant. The method provides the ruminant one or more feed ingredients, and provides the ruminant an α-amino acid analog selected from the group consisting of 2-hydroxy-4-(methylthio)butanoic acid, and salts, esters, amides, ethers, diesters, ester/ethers, oligomers, metal chelates, and anion salts thereof; and the salt, ester, amide, ether, oligomer, metal chelate, and anion salt analogs of methionine. The assortment and composition of said feed ingredients are such that the amounts thereof which can be consumed by the growing ruminant in one day can collectively satisfy the ruminant's daily nutrient requirements, and exceed its daily maintenance energy requirements, provided that such assortment and composition may not necessarily satisfy the growing ruminant's methionine requirements. The salts of 2-hydroxy-4-(methylthio)butanoic acid are selected from the group consisting of ammonium, magnesium, lithium, sodium, potassium, and zinc.

The present invention is further directed to a method of improving weight gain of a growing ruminant comprising providing the ruminant one or more feed ingredients and providing the ruminant an α-amino acid analog selected from the group consisting of the calcium salt of 2-hydroxy-4-(methylthio)butanoic acid. The combination of feed ingredients comprise at least 80% forage. The assortment and composition of said feed ingredients are such that the amounts thereof which can be consumed by the growing ruminant in one day can collectively satisfy the ruminant's daily nutrient requirements, and exceed its daily maintenance energy requirements, provided that such assortment and composition may not necessarily satisfy the growing ruminant's methionine requirements.

The present invention is further directed to a process of formulating a feed ration for growing ruminants. In this process the nutrient and maintenance energy needs of the growing ruminant are determined. One or more feed ingredients is identified and the nutrient and energy contribution of each of said feed ingredients other than α-amino acid analogs is determined. A feed ration from the identified feed ingredients such that the amount of feed ration which can be consumed by the growing ruminant in one day can collectively satisfy the ruminant's daily nutrient requirements, and exceed its daily maintenance energy requirements, provided that such assortment and composition may not necessarily satisfy the growing ruminant's methionine requirements. An α-amino acid analog is additionally incorporated into the feed ration without regard to its energy contribution to the feed ration.

The present invention is further directed to a feed ration for growing ruminants comprising one or more feed ingredients and an α-amino acid analog. The feed ration comprises at least 50% forage. The α-amino acid analog is selected from the group consisting of 2-hydroxy-4-(methylthio)butanoic acid, and salts, esters, amides, ethers, diesters, ester/ethers, oligomers, metal chelates, and anion salts thereof; and the salt, ester, amide, ether, oligomer, metal chelate, and anion salt analogs of methionine. The salts of 2-hydroxy-4-(methylthio)butanoic acid being selected from the group consisting of ammonium, magnesium, lithium, sodium, potassium, and zinc. The feed ration being formulated such that an amount of feed ration that is capable of being consumed by a growing ruminant in a day satisfies the ruminant's daily nutritional requirements, and exceeds its daily maintenance energy requirements; provided that such assortment and composition may not necessarily satisfy the growing ruminant's methionine requirements.

The present invention is further directed to a feed ration for growing ruminants comprising one or more feed ingredients and the calcium salt of 2-hydroxy-4-(methylthio)butanoic acid. The feed ration being formulated such that an amount of feed ration that is capable of being consumed by a growing ruminant in a day satisfies the ruminant's daily nutritional requirements, and exceeds daily maintenance energy requirements of a growing ruminant; provided that such assortment and composition may not necessarily satisfy the growing ruminant's methionine requirements.

Other features of the present invention will be in part apparent to those skilled in the art and in part pointed out in the detailed description provided below.

### BRIEF DESCRIPTION OF DRAWINGS

FIG. 1 is a graph illustrating the relationship between dry matter intake and daily weight gain for each pen of growing cattle during a 112-day ruminant feed trial.

### DETAILED DESCRIPTION OF THE PREFERRED EMBODIMENTS

The present invention relates to feed rations for growing ruminants, processes of formulating feed rations for growing ruminants, and methods of improving weight gain in a growing ruminant wherein the ruminant is provided a feed ration comprising one or more feed ingredients and further comprising an analog of an α-amino acid. Preferably, the form, manner, schedule and circumstances in which said feed ingredients are provided are effective to induce the ruminant to consume an amount of nutrients that provides at least enough energy required for the ruminant to maintain its weight (i.e., satisfy the ruminant's maintenance energy requirements). Once enough nutrients are consumed by the ruminant to satisfy the ruminant's maintenance energy requirements, the consumed α-amino acid analog beneficially improves the weight gain of the ruminant.

A ruminant utilizes energy derived from feed ingredients it consumes to perform bodily functions such as maintaining its body temperature, carrying out physiological and metabolic processes, conducting physical activity, and the like, as well as promote weight gain. A ruminant's “maintenance energy requirements” is the amount of energy that is required by a ruminant to carry out its bodily functions without resulting in a gain or loss of body tissue. Once enough nutrients are consumed by the ruminant to meet its maintenance energy requirements, additional energy derived from consumed feed ingredients may be utilized for production (i.e., weight gain). Consumption of feed ingredients which provide energy in excess of a ruminant's maintenance energy requirements permits the ruminant to increase its body fat, body protein, and body weight.

Ruminants will consume feed ingredients until their energy requirements are satisfied. The amount of feed that a ruminant can consume, however, is limited to the physical size of the ruminant and its rumen. Typically, a growing ruminant can consume between about 2% to about 3% of its own weight in dry matter per day. For example, a 500 pound ruminant can consume approximately 10 to 15 pounds of dry matter a day. In determining the amount of feed consumed, the dry weight of a feed source is used.

When formulating feed rations for ruminants, the quality of the feed ingredients selected for a feed ration is significant. If a feed source is too poor in quality (i.e., it contains low levels of nutrients or is difficult to digest), the amount of the feed ingredient necessary to satisfy a ruminant's energy requirements may exceed the physical feed consumption capacity of the ruminant. For example, if a ruminant is offered only poor quality forage feed ingredients wherein 17 pounds of the forage would be required to provide enough energy to meet a 500 pound ruminant's maintenance energy requirements, the ruminant would be unable to consume enough forage to satisfy its maintenance energy requirements. In such a case, the ruminant would not be able to gain weight without consuming an alternative higher quality feed ingredient.

The nutrient and energy content of many common ruminant feed ingredients have been measured and are available to the public. The National Research Council has published a book that contains tables of common ruminant feed ingredients and their respective measured nutrient and energy content. Additionally, estimates of nutrient and maintenance energy requirements are provided for growing and finishing cattle according to the weight of the cattle. National Academy of Sciences, Nutrient Requirements of Beef Cattle, Appendix Tables 1-19, 192-214, (National Academy Press, 2000), incorporated herein in its entirety. This information can be utilized by one skilled in the art to estimate the nutritional and maintenance energy requirements of growing cattle and determine the nutrient and energy content of ruminant feed ingredients.

The applicants have discovered that growing ruminants will have improved weight gain if they consume a feed ration comprising feed ingredients that collectively contribute enough nutrients and energy to satisfy the ruminant's nutrient requirements, or are deficient in methionine but otherwise satisfy the ruminant's daily nutritional requirements, and exceed their maintenance energy requirements if the ruminants additionally consume an amount of an α-amino acid analog. However, if the feed ingredients do not contribute enough energy to at least meet the maintenance requirements of a growing ruminant, the additional consumption of an α-amino acid analog will not typically result in improved weight gain.

Feed Ingredients

Feed ingredients that may be utilized in the present invention to satisfy a ruminant's maintenance energy requirements may include feed ingredients that are commonly provided to ruminants for consumption. Examples of such feed ingredients include forage, grain, feed meals, feed concentrates, vitamins, minerals, and the like. While feed rations and methods of the present invention provide “α-amino acid analogs” to ruminants as part of their feed ration, references to “feed ingredients,” as used herein, refer to feed ingredients other than “α-amino acid analogs.”

Forage products are feed ingredients such as vegetative plants in either a fresh (pasture grass or vegetation), dried, or ensiled state and may incidentally include minor proportions of grain (e.g., kernels of corn that remain in harvested corn plant material after harvest). Forage includes plants that have been harvested and optionally fermented prior to being provided to ruminants as a part of the feed of the present invention. Thus, forage includes hay, haylage, and silage. Examples of hay include harvested grass, either indigenous to the location of the ruminants being fed or shipped to the feeding location from a remote location. Non-limiting examples of hay include alfalfa, Bermuda grass, bahia grass, limpo grass, rye grass, wheat grass, fescue, clover, and the like as well as other grass varieties that may be native to the location of the ruminants being provided the ruminant feed ration.

It is beneficial if the forage is relatively high quality (i.e., contains relatively levels of metabolizable nutrients which permit the ruminant to satisfy its nutrient and maintenance energy requirements before reaching its consumption capacity). If the forage is of low quality, the ruminant may not metabolize it adequately to achieve desired performance effects (e.g., satisfy its nutrient and/or maintenance energy requirements), not only compromising the nutritional benefit from the forage per se, but also causing the ruminant to feel full or bloated, and possibly deterring it from consuming sufficient nutrients and α-amino acid analog.

Haylage is a forage product that has been naturally fermented by harvesting a hay crop while the sap is still in the plant. The harvested hay or hay bales are then stored in an air-tight manner in which fermentation can occur. The fermentation process converts the sugars in the plants into acids which lower the pH of the harvested hay and preserves the forage.

Silage, similar to haylage, is a forage product that is produced from the harvest, storage and fermentation of green forage crops such as corn and grain sorghum plants. These crops are chopped, stems and all, before the grain is ready for harvest. The plant material is stored in silos, storage bags, bunkers or covered piles causing the material to ferment, thereby lowering the pH and preserving the plant material until it can be fed.

Forage products also include high fiber sources and scrap vegetation products such as green chop, corncobs, plant stalks, and the like.

Grain products include corn, corn gluten meal, soybeans, soybean meal, wheat, barley, oats, sorghum, rye, rice, and other grains and grain meals.

Feed concentrates are ruminant feedstuffs that are high in energy and low in crude fiber. Concentrates also include a source of one or more ingredients that are used to enhance the nutritional adequacy of a feed supplement mix, such as vitamins and minerals.

Other ingredients may be optionally included in the ruminant feed to provide additional nutrients to the ruminants. Examples of optional ingredients include urea, fat, vitamins, minerals, and the like. Urea provides rumen bacteria a source of non-protein nitrogen from which they are able to synthesize bacterial protein. These ingredients may also be excluded as necessary to provide a feed ration to ruminants that can be tailored to meet their nutritional needs.

α-Amino Acid Analogs

Ruminants possess multi-chambered stomachs. The largest of these chambers, the rumen, contains bacteria that are active in degrading a significant amount of ingested protein and amino acids. For the ruminant to utilize ingested protein and amino acids, the protein and amino acids must be in a form that is available for absorption by the ruminant and not completely degraded by rumen bacteria. Protein and amino acids that are available for absorption by the ruminant are absorbed through the rumen wall and other parts of the digestive tract such as the abomasum, omasum, and small intestine.

Chemical analogs of α-amino acids, when ingested, have been found to resist bacterial degradation to a greater extent than α-amino acids in their natural forms. Once absorbed or present in the abomasum, omasum, or small intestine, the α-amino acid analogs are converted by biochemical reactions within the ruminant's body to an α-amino acid form that can be utilized by the ruminant as an amino acid supplement (e.g., methionine supplement, lysine supplement, and the like) to meet its amino acid nutritional needs.

As used herein, an “α-amino acid analog” is any compound having an α-amino acid structure wherein either the α-amine moiety, the carboxy acid group, or both have been substituted with an ester, amide, ether, or hydroxy group. An “α-amino acid analog” may also include oligomers or polymers of an α-amino acid or oligomers or polymers of an ester, amide, or ether of an α-amino acid. Furthermore, an “α-amino acid analog” may also include salts, metal chelates, and anion salts of an α-amino acid analog. Thus, amino acid supplements that may be used in the process of the present invention include the amide, ester, ether, and oligomer analogs of an α-amino acid and salts, metal chelates, and anion salts thereof (hereinafter collectively referred to as “α-amino acid analogs”). While “α-amino acid analogs” have nutritional value and are incorporated into the feed ration of the present invention, references to “nutrients” or “feed ingredients,” as used herein, refer to nutrients and feed ingredients other than “α-amino acid analogs.”

The α-amino acid analog ingredient of the ruminant feed may be a chemical analog for any of the naturally occurring α-amino acids, e.g., asparagine, glycine, alanine, valine, leucine, isoleucine, phenylalanine, proline, serine, threonine, cysteine, methionine, tryptophan, tyrosine, glutamine, aspartic acid, glutamic acid, lysine, arginine, and histidine.

In one embodiment, the α-amino acid analog includes one or more supplements for essential amino acids, i.e., isoleucine, phenylalanine, leucine, lysine, methionine, threonine, tryptophan, histidine and valine. In a particular embodiment, the α-amino acid analog includes amino acid supplements for methionine and/or lysine.

The α-amino acid analogs may comprise an α-hydroxy analog of an α-amino acid, its salts, esters, amides, ethers, diesters, ester/ethers, oligomers, metal chelates, and anion salts thereof.

Representative salts of an α-hydroxy analog of an α-amino acid include the ammonium, magnesium, calcium, lithium, sodium, potassium, and zinc salts. Representative esters of HMBA include the methyl, ethyl, n-propyl, isopropyl, butyl esters, namely n-butyl, sec-butyl, isobutyl, and tertiary butyl esters, pentyl esters, namely n-pentyl and isopentyl esters, and hexyl esters, namely -hexyl and isohexyl esters. Representative amides corresponding to 2-hydroxy-4-(methylthio)butanoic acid include methylamide, dimethylamide, ethylmethylamide, butylamide, dibutylamide, butylmethylamide, and alkyl ester of N-acyl methionates (e.g., alkyl N-acetyl methioninates. Representative ethers include methyl, ethyl, n-propyl, isopropyl, butyl ether, namely n-butyl, sec-butyl, isobutyl, and tertiary butyl ethers, pentyl ethers, namely n-pentyl and isopentyl ethers, and hexyl ethers, namely -hexyl and isohexyl ethers of HMBA. Representative oligomers of HMBA include its dimers, trimers, tetramers and oligomers which include a greater number of repeating units. Oligomers of HMBA may also include oligomers of HMBA formed from combining HMBA with one or more amino acid residues. The HMBA-amino acid oligomers are described by Lorbert et al. in U.S. application Ser. No. 09/699,946 at page 8, line 19 to page 11, line 24 and Lorbert in U.S. application Ser. No. 10/136,974 at page 9, line 27 to page 13, line 20. Both Lorbert et al. applications are incorporated by reference herein in their entirety. Representative metal chelates and anion salts of HMBA include HMBA chelates and anion salts of a bivalent metal, including zinc, copper, cobalt, manganese, calcium, iron, or magnesium. The molar ratio of HMBA to bivalent metals include between about 0.5:1 to about 5:1.

In one embodiment, the α-hydroxy analog of an α-amino acid analog comprises 2-hydroxy-4-(methylthio)butanoic acid (HMBA).

In another embodiment, the α-hydroxy analog of an α-amino acid analog comprises the isopropyl ester of 2-hydroxy-4-(methylthio)butanoic acid (HMBi).

In another embodiment, the α-hydroxy analog of an α-amino acid analog comprises a zinc chelate wherein the molar ratio of HMBA to zinc is between about 1.5:1 to about 2.5:1, more preferably the molar ratio of HMBA to zinc is about 2:1.

In another embodiment, the α-hydroxy analog of an α-amino acid analog comprises an zinc anion salt wherein the molar ratio of HMBA to zinc is between about 0.5:1 to about 1.5:1, more preferably the molar ratio of HMBA to zinc is about 1:1.

In another embodiment, the α-hydroxy analogs of an amino acid are selected from the group of 2-hydroxy-4-(methylthio)butyric acid, and isopropyl ester or tertiary butyl ester thereof.

In another embodiment, the α-amino acid analog comprises the calcium salt of 2-hydroxy-4-(methylthio)butanoic acid (HMBA).

The α-amino acid analog may also be provided as the salt, ester, amide, ether, oligomer analogs, metal chelate and anion salt of an α-amino acid. In one embodiment, the α-amino acid analog is an analog of methionine.

Representative salts of methionine include the ammonium, magnesium, calcium, lithium, sodium, potassium, and zinc salts. Representative esters of methionine include the methyl, ethyl, n-propyl, isopropyl, butyl esters, namely n-butyl, sec-butyl, isobutyl, and tertiary butyl esters, pentyl esters, namely n-pentyl and isopentyl esters, and hexyl esters, namely -hexyl and isohexyl esters of methionine. Representative amides of methionine include methylamide, dimethylamide, ethylmethylamide, butylamide, dibutylamide, butylmethylamide, alkyl ester of N-acyl methionates (e.g., alkyl N-acetyl methionates). Representative ethers of methionine include the methyl, ethyl, n-propyl, isopropyl, butyl ether, namely n-butyl, sec-butyl, isobutyl, and tertiary butyl ethers, pentyl ethers, namely n-pentyl and isopentyl ethers, and hexyl ethers, namely n-hexyl and isohexyl ethers of HMBA. Representative oligomers of methionine include its dimers, trimers, tetramers and oligomers which include a greater number of repeating units. Representative metal chelates and anion salts of methionine include methionine chelates and anion salts of a bivalent metal, including zinc, copper, cobalt, manganese, calcium, iron, or magnesium. The molar ratio of methionine to bivalent metals include between about 0.5:1 to about 5:1.

In one embodiment, the α-amino acid analogs are selected from the group of the isopropyl ester and tertiary butyl ester of methionine.

In another embodiment, the α-amino acid analog comprises a zinc chelate of methionine wherein the molar ratio of methionine to zinc is between about 1.5:1 to about 2.5:1, more preferably the molar ratio of methionine to zinc is about 2:1.

In another embodiment, the α-amino acid analog comprises an zinc anion salt of methionine wherein the molar ratio of methionine to zinc is between about 0.5:1 to about 1.5:1, more preferably the molar ratio of methionine to zinc is about 1:1.

In another embodiment, the α-amino acid analog may be provided as a lysine supplement. The lysine supplement includes the salts, esters, amides, ethers, and oligomers of lysine. Representative salts of lysine include the ammonium, magnesium, calcium, lithium, sodium, potassium, and zinc salts. Representative esters of lysine include the methyl, ethyl, n-propyl, isopropyl, butyl esters, namely n-butyl, sec-butyl, isobutyl, and tertiary butyl esters, pentyl esters, namely n-pentyl and isopentyl esters, and hexyl esters, namely n-hexyl and isohexyl esters of lysine. Representative amides of lysine include methylamide, dimethylamide, ethylmethylamide, butylamide, dibutylamide, butylmethylamide, alkyl ester of N-acyl lysinates (e.g., alkyl N-acetyl lysinates). Representative ethers of lysine include the methyl, ethyl, n-propyl, isopropyl, butyl ether, namely n-butyl, sec-butyl, isobutyl, and tertiary butyl ethers, pentyl ethers, namely n-pentyl and isopentyl ethers, and hexyl ethers, namely n-hexyl and isohexyl ethers of lysine. Representative oligomers of lysine include its dimers, trimers, tetramers and oligomers which include a greater number of repeating units.

Feed Ration

In accordance with the present invention, it has been discovered that the weight gain of a growing ruminant can be improved if a growing ruminant is provided a feed ration that satisfies the ruminant's nutrient needs or satisfies the ruminant's nutrient needs but are deficient in methionine and which also supplies enough energy to exceed the ruminant's maintenance energy requirements if the growing ruminant is also provided an α-amino acid analog.

The concentration of α-amino acid analog in a mixture of a feed ingredient and α-amino acid analog should be low enough so that the mixture remains substantially palatable and the ruminant is naturally drawn to consume at least an amount feed ingredients to satisfy its daily maintenance energy requirements and additionally consume at least about 0.1 gram per day of an α-amino acid analog.

In one embodiment, the form of the ration is such that, if provided in a satisfactory manner, schedule and circumstances as described elsewhere herein, the ruminant will consume between about 0.1 and about 50 grams of α-amino acid analog per day, preferably the ruminant will consume between about between about 1 and about 25 grams of α-amino acid analog per day, preferably, the ruminant will consume between about 5 and about 20 grams of α-amino acid analog per day, more preferably, the ruminant will consume between about 10 and about 18 grams of α-amino acid analog per day.

Forage feed ingredients may sometimes have a low nutritional content or may be deficient in sufficient methionine levels necessary to meet a ruminant's nutrient and maintenance energy requirements. In some instances, the forage feed is so deficient in nutrients that the amount of forage required to satisfy a ruminant's nutritional and energy requirements exceeds the consumption capacity of the ruminant (i.e., a ruminant physically cannot eat enough forage to obtain its required nutrients).

In order to gain weight, growing ruminants must consume enough forage and other feed ingredients to meet their nutrient requirements and exceed their maintenance energy requirements. If the forage and other feed ingredients contain just enough nutrients to meet or slightly exceed the ruminants' nutrient and maintenance requirements, or if the feed ingredients are deficient in methionine, the growing ruminant will not gain weight or will only slowly gain weight. Thus, the method of the present invention can improve weight gain of growing ruminants wherein forage ingredients account for a significant percentage of the feed ration consumed by the growing ruminant. By formulating a feed ration within the consumption capacity of a ruminant which supplies adequate nutrients to satisfy its nutritional and energy needs wherein an α-amino acid analog is additionally provided to satisfy its methionine needs, weight gain of the growing ruminant will be improved. In one embodiment, the feed ration of the present invention comprises at least 50% forage and an α-amino acid analog, preferably the feed ration comprises at least 65% forage and an α-amino acid analog, preferably the feed ration comprises at least 80% forage and an α-amino acid analog, preferably the feed ration consumed by the growing ruminants comprise at least 85% forage and an α-amino acid analog.

In one embodiment, the feed ration comprises molasses. Molasses is a feed ingredient that provides energy to the ruminant as well as providing an energy and carbon source to the rumen bacteria for the beneficial synthesis of bacterial protein. Molasses is a byproduct of sugar refining and is available in a variety of forms that depend upon the starting materials that are refined. The most common form of molasses in the U.S. is cane molasses which is the by-product of the manufacture or refining of sucrose from sugar cane. Beet molasses is also a common form of molasses which is a by-product of the manufacture or refining of sucrose from sugar beets. Other forms of molasses include citrus molasses, formed from the partially dehydrated juices obtained from the manufacture of dried citrus pulp, and starch molasses, which is a by-product of the manufacture of dextrose from starch derived from corn or grain sorghums where the starch is hydrolyzed by enzymes and/or acid.

The molasses and α-amino acid analog ingredients may be mixed by hand or by an automated mixing process to evenly disperse the α-amino acid analog within the molasses. Even dispersion of the α-amino acid analog in the molasses is desirable to ensure that ruminants consume the desired amount of α-amino acid analog. Furthermore, even mixing of the ingredients helps to prevent areas of high α-amino acid analog concentration in the mixture which would cause a ruminant to refuse to consume the mixture.

In one embodiment, the amount of molasses made available to the ruminant in the feed ration comprises at least about 0.5 lbs/day, preferably, the feed ration comprises at least about 1 lb/day, more preferably, the feed ration comprises at least about 2 lbs/day.

In another embodiment, the amount of molasses provided in the feed ration is between about 1 and about 7 lbs/day, preferably, the amount of molasses provided in the feed ration is between about 2 and about 6 lbs/day, more preferably, the amount of molasses provided in the feed ration is between about 3 and about 5 lbs/day.

In another embodiment, a feed ration is provided which comprises at least 50% forage and further comprises molasses wherein the ratio of forage to molasses feed ingredients is between about 20:1 to about 1.5:1, preferably, the ratio of forage to molasses feed ingredients in a ratio between about 10:1 to about 2:1, more preferably, the ratio of forage to molasses feed ingredients is between about 7:1 to about 2.5:1.

In another embodiment of the present invention, a method of improving weight gain of a growing ruminant is utilized wherein the nutritional and maintenance energy requirements of the growing ruminant are determined. The ruminant is provided one or more feed ingredients wherein the assortment and composition of said feed ingredients are such that the amounts thereof which can be consumed by the growing ruminant in one day can collectively satisfy the ruminant's daily nutrient requirements and exceed its daily maintenance energy requirements, provided that such assortment and composition may not necessarily satisfy the growing ruminant's methionine requirements. Additionally, an α-amino acid analog is provided to the ruminant.

In another embodiment, a method of improving weight gain of a growing ruminant is utilized wherein the nutritional and maintenance energy requirements of the growing ruminant are determined. The ruminant is provided one or more feed ingredients wherein the wherein the assortment and composition of said feed ingredients are such that the amounts thereof which can be consumed by the growing ruminant in one day does not satisfy the ruminant's methionine requirements, but otherwise satisfies the ruminant's daily nutrient requirements and exceeds its daily maintenance energy requirements. Additionally, an α-amino acid analog is provided to the ruminant.

In another embodiment, a daily feed ration is formulated wherein about 4 pounds to about 30 pounds of the feed ration satisfies the nutrient requirements and exceeds the maintenance energy requirements for growing ruminants weighing between about 200 and about 1000 pounds. In a preferred embodiment, a daily feed ration is formulated wherein about 4 pounds to about 12 pounds of the feed ration satisfies the nutrient requirements and exceeds the maintenance energy requirements for growing ruminants weighing between about 200 and about 400 pounds. In another preferred embodiment, a daily feed ration is formulated wherein about 8 pounds to about 24 pounds of the feed ration satisfies the nutrient requirements and exceeds the maintenance energy requirements for growing ruminants weighing between about 400 and about 800 pounds.

In another embodiment, a daily feed ration is formulated wherein about 4 pounds to about 30 pounds of the feed ration is deficient in methionine, but otherwise satisfies the nutritional requirements, and exceeds the maintenance energy requirements for growing ruminants weighing between about 200 and about 1000 pounds. In a preferred embodiment, a daily feed ration is formulated wherein about 4 pounds to about 12 pounds of the feed ration is deficient in methionine, but otherwise satisfies the nutritional requirements, and exceeds the maintenance energy requirements for growing ruminants weighing between about 200 and about 400 pounds. In another preferred embodiment, a daily feed ration is formulated wherein about 8 pounds to about 24 pounds of the feed ration is deficient in methionine, but otherwise satisfies the nutritional requirements and exceeds the maintenance energy requirements for growing ruminants weighing between about 400 and about 800 pounds.

The feed rations of the present invention can also be formulated to provide α-amino acid analogs to supplement specific α-amino acid needs of a ruminant. When formulating such a ration to meet the ruminants' nutritional needs, the amount of α-amino acid analog which is not degraded by rumen bacteria and therefore available for absorption must be considered. Methionine and lysine are respectively the first and second most limiting amino acids for cattle. The lysine to methionine ratio typically required by a ruminant to satisfy its essential amino acid requirements is approximately 3:1. As an example, a feed ration may be formulated to provide lysine and methionine supplements in amounts such that the supplements are available for absorption by the ruminant in a lysine to methionine supplement ratio of 3:1 when rumen degradation of the supplements is taken into account. Thus, the combination of α-amino acid analog supplements can be formulated to meet the essential amino acid requirements of a ruminant for lysine and methionine.

A feed ration of the present invention may be formulated to meet the essential amino acid requirements of a particular ruminant by incorporating two or more different α-amino acid analogs (e.g., methionine and lysine supplements) into the feed which are limiting for the ruminant. The α-amino acid analogs can be included in proportions that fulfill the ruminant's nutritional requirements for the particular amino acids.

In one embodiment, a feed ration is formulated to provide lysine and methionine supplements in amounts such that the supplements are available for absorption by the ruminant in a lysine to methionine supplement ratio of about 1:1 to about 5:1 when rumen degradation of the supplements is taken into account. More preferably, the feed ration is formulated to provide lysine and methionine supplements in amounts such that the supplements are available for absorption by the ruminant in a ratio of about 2:1 to about 4:1 when rumen degradation of the supplements is taken into account.

The feed ration of the present invention should be made available to the ruminant in a form, manner, schedule, and circumstances that are effective to induce the ruminant to consume a daily amount of feed ingredients within the ruminant's consumption capacity that satisfies its nutritional and maintenance energy requirements and additionally induces it to consume an amount of an α-amino acid analog.

Growing Ruminants

As discussed above, ruminants are animals that possess multi-chambered stomachs, the largest of which is the rumen. Examples of growing ruminants include beef and dairy calves, young steers, heifers, cows and cattle, sheep, and goats. In one embodiment, growing ruminants include beef and dairy calves that have been weaned or which are still nursing but also consume solid feed, such as grain and forage, young beef or dairy cattle that have not reached maturity, and beef or dairy cattle that have not attained average adult weight level ranges for their sub-species.

In one embodiment, the method of the present invention is utilized to promote weight gain of growing beef and dairy cattle wherein the individual weight of growing cattle is generally less than about 1000 pounds, preferably, the individual weight of growing cattle is between about 200 pounds and about 1000 pounds. Growing cattle weighing between about 200 pounds and about 1000 pounds will typically consume between about 4 pounds to about 30 pounds of food per day.

In one embodiment, the method of the present invention is utilized to promote weight gain of growing cattle that are “creep fed” calves wherein the individual weight of the calves is between about 200 pounds and about 400 pounds. Creep-fed calves are calves that are in a transition stage in the type of feed they ingest wherein they are not weaned or are in the process of being weaned, but are also beginning to receive nutrients from sources other than milk such as grain and forage feed. In a preferred embodiment, creep-fed calves are provided feed ingredients and an α-amino acid analog in addition to the nutrients they obtain from consuming milk. Growing cattle weighing between about 200 pounds and about 400 pounds will typically consume between about 4 pounds to about 12 pounds of food per day.

In another embodiment, the method of the present invention is utilized on growing cattle wherein the individual weight of growing cattle is between about 400 pounds and about 1000 pounds. Growing cattle weighing between about 400 pounds and about 1000 pounds will typically consume between about 8 pounds to about 30 pounds of food per day.

Preferably, the method of the present invention is utilized on growing cattle wherein the individual weight of growing cattle is between about 400 pounds and about 800 pounds. In the cattle industry, the 400 to 800 pound weight range is significant in that it is a growth stage wherein rapid growth is highly desirable. It is typically the stage at which a growing calf has been weaned and is obtaining the nutrients it needs to grow from forage and grain sources. Industry practice commonly provides growing cattle in this weight range a feed ration that contains a high percentage of forage, e.g., the growing cattle are provided harvested forage or are required to graze on pasture grass. Growing cattle weighing between about 400 pounds and about 800 pounds will typically consume between about 8 pounds to about 24 pounds of food per day.

Feeding Methods

The feed ration may be provided in a variety of forms. The feed ingredient and α-amino acid analog may be made available to the ruminants as separate ingredients. In another form, the feed ration may be provided in a mixture of two or more feed ingredient ingredients and an α-amino acid analog. The feed ingredient and α-amino acid analog ingredients may be mixed by hand or machine mixer. In such a form, the α-amino acid analog may be mixed or dissolved in the feed ingredient. The harvested forage may be mixed in the same form in which it is harvested or, alternatively, first chopped or processed into smaller pieces and mixed with an α-amino acid analog, thereby causing any consumption of the α-amino acid analog and harvested forage to be simultaneous. Alternatively, the α-amino acid analog may be applied to a forage or other feed ingredient as a top dressing.

In one embodiment of the present invention, a feed ration is formulated to comprise a combination of feed ingredients and an α-amino acid analog. The feed ingredients and the α-amino acid analog may be mixed together or provided separately but contemporaneously, i.e., made accessible, to the ruminant. However, the α-amino acid analog is generally not provided separately from forage or other feed ingredients because most α-amino acid analogs are malodorous or otherwise unpalatable to the ruminant. Where the α-amino acid analog source is incorporated in the forage or other feed ingredients, a high concentration of the α-amino acid analog may render the entire feed ingredient unpalatable.

The α-amino acid analog concentration of the mixture above which the ruminant will refuse to consume the mixture varies depending on a variety of factors. These factors include the type of forage or other feed ingredients selected, the α-amino acid analog selected, and the particular ruminant being fed the mixture. Thus, to induce the ruminant to ingest at least 0.1 grams of α-amino acid analog per day and further consume forage in quantities effective for weight gain, it is necessary that the form, manner, schedule and circumstance under which the feed ration is provided be controlled to avoid rejection or undue limitation on consumption of any nutrient component of the ration.

In one embodiment, the α-amino acid analog concentration in a mixture of a feed ingredient and α-amino acid analog is between about 0.01% to about 2.0%, preferably, the α-amino acid analog is between about 0.1% to about 1.5%, preferably, the α-amino acid analog is between about 0.2% to about 1.0%.

The manner in which the forage or other feed ingredients and α-amino acid analog are made available to ruminants may include providing individual ingredients separately available. An example of which would include harvested forage being placed in a feeding ring, other feed ingredients such as grain, molasses, concentrates, and the like being provided in a trough or lick, and the α-amino acid analog also being made available in a separate dispensing lick. The manner may also include making available a mixture of two or more of the ingredients. An example of the manner in which mixtures may be made available include placing a mixture of a feed ingredient and α-amino acid analog in a trough, dispensing feeder or lick, and the harvested forage could be separately made available in a hay ring. In one embodiment, a mixture of a feed ingredient and an α-amino acid analog is provided in a trough, dispensing feeder, or lick, and harvested forage is separately made available in a hay ring or as pasture grass.

In another embodiment, a mixture of a feed ingredient and an α-amino acid analog, whether it is harvested forage top-dressed with an α-amino acid analog or chopped harvested forage mixed with an α-amino acid analog, may be made available in a trough, feed ring, simply placed on the ground, and the like.

The schedule in which the forage or other feed ingredient and an α-amino acid analog are made available may be based on a variety of considerations. Such considerations may include the availability of labor, the degree of automation, the eating characteristics of the ruminant, and the like. The schedule may also be altered based on observations of the ruminants consuming the ingredients. One or more ingredients may be made available ad libitum. An example of this includes providing an excess of harvested forage in a feed ring and an excess of one or more feed ingredient and an α-amino acid analog being made available for the ruminants to consume in a feeding trough or container. Alternatively, one or more ingredients may be made available ad libitum while one or more ingredients is made available in a manner that is limiting. An example includes providing an excess of harvested forage in a feed ring and limit feeding a specific amount of a mixture of another feed ingredient and α-amino acid analog. The mixture may be provided to ruminants in a variety of manners including placement in a feeding trough in proportions wherein the ruminants consume a desired amount of a feed ingredient and an α-amino acid analog per day. Alternatively, the mixture may be made available to the ruminants on alternative days or similar feeding schedule. Alternatively, the mixture may be dispensed to the ruminants in a demand feeder, controlled release feeder, or similar container. By utilizing a controlled release feeder, the feed ingredient and α-amino acid analog may be dispensed at a rate that provides the ruminants with the amount of feed ingredients and α-amino acid analog that is calculated to meet their maintenance energy requirements and nutritional needs.

Intake of the feed ration may also be self-controlled by the ruminant. One means this may be achieved is through lowering the pH of a mixture of a feed ingredient and α-amino acid analog. A lower pH causes a ruminant to limit the amount of feed ingredient and α-amino acid analog mixture it consumes in a day. Another means includes controlling intake by adding salt to a mixture of a feed ingredient and α-amino acid analog. The intake of salt in the mixture also causes the ruminant to limit the amount of mixture it consumes in a day.

Intake can also be promoted by mixing the α-amino acid analog with a sweet ingredient, such as molasses and providing either separately in a lick, dispenser, trough, and the like or by mixing with forage and other feed ingredients. The concentration of α-amino acid analog contained in the sweet ingredient is such that the growing ruminant will be induced to consume between about 0.1 gram to about 50 grams of α-amino acid analog per day, preferably between about 1 gram to about 25 grams of α-amino acid analog per day, more preferably between about 5 grams to about 20 grams of α-amino acid analog per day, still more preferably, between about 10 grams to about 18 grams of α-amino acid analog per day.

In another embodiment, the α-amino acid analog is provided in a salt lick while forage or other feed ingredient is provided separately.

Circumstances wherein ruminants are induced to consume the feed ingredients are dependent on the a variety of factors. Examples of some of these factors include the quality of forage or other feed ingredient (e.g., the carbohydrate and protein content) which the ruminant consumes; the α-amino acid analog selected; the α-amino acid content of other ingredients which the ruminant consumes, the amino acid requirements of a ruminant based on its sex, weight, and condition (e.g., young calf, growing ruminant, pregnant ruminant, lactating ruminant, and the like), and environmental conditions and stress (e.g., environmental temperature).

The circumstances in which the ruminant feed is made available may be related to a variety of factors including the desired rate of weight gain sought to be attained. By identifying the rate of weight gain to be achieved, a farmer or rancher may increase or decrease the amount of forage or other feed ingredient and α-amino acid analog made available according to observations of whether the rate of weight gain is being achieved. For example, environmental circumstances such as either significant availability of pasture forage during the spring and summer months may be a circumstance wherein the ruminants do not need the additional nutrients that the ruminant feed can provide. In winter months, when pasture forage is scarce, ruminant feed ingredients may be made available in greater quantities to provide nutrients the ruminants need to satisfy their maintenance energy requirements and continue to grow as well as withstand potentially harsher winter weather.

Computer models are available which may be utilized to formulate ruminant rations in a manner that determines the ruminants nutrient needs based upon the ingredients it is fed. Methods of formulating a ruminant feed ration wherein hydroxy analogs of methionine are provided as methionine supplements are disclosed in Knight et al., U.S. Pat. No. 6,017,563, beginning at column 3, line 5. U.S. Pat. No. 6,017,563 is incorporated herein in its entirety.

Definitions

“Consumption capacity” as used herein refers to the total amount of dry matter that a growing ruminant is capable of ingesting in a single day. For example, the consumption capacity for growing cattle is between about 2% to about 3% of the body weight of the growing cattle.

“HMBA” as used herein refers to 2-hydroxy-4-(methylthio)butanoic acid.

“HMBi” as used herein refers to the isopropyl ester of 2-hydroxy-4-(methylthio)butanoic acid.

The following examples illustrate the invention.

### EXAMPLE 1

### Method of Maintaining or Increasing Weight of Ruminants

An experiment was conducted to study the effect of increasing quantities of methionine from corn gluten meal, an 88% free acid solution of 2-hydroxy-4-(methylthio)butanoic acid sold under the trademark Alimet® (Novus International, Inc., St. Louis, Mo.), on the performance of growing beef cattle fed bermuda grass hay supplemented with molasses-based supplements. The compositions of the tested supplements are provided in Table 1. The experiment evaluated the effects of adding corn gluten meal or Alimet® to diets of hay supplemented with molasses-based supplements fed to growing beef cattle.

### Procedures

This experiment evaluated corn gluten meal and Alimet® fed to growing beef cattle at four levels to provide 0, 2, 4, and 6 gm/day of added bypass total sulfur amino acids (TSAA) assuming a 40% bypass of Alimet®. An eighth treatment was formulated to provide 8 gm/day of added bypass TSAA from Alimet®. Both TSAA sources were provided in a liquid supplement slurry containing 82% fortified molasses (fortified with ‘12% crude protein, minerals, vitamins) and 18% ground corn or corn gluten meal. A summary of formulations is provided in Table 1. Corn gluten meal replaced corn in the supplement keeping the proportion of dry ingredients in the supplement similar across TSAA sources and levels. All supplements were offered to growing cattle to provide 3.28 lb/day of total digestible nutrients (TDN). Supplements were formulated to provide adequate nitrogen in the form of rumen degradable intake protein (DIP) with 0.18 lb DIP provided for each pound of TDN. Much of the DIP in supplements was provided by urea. Ingredient compositions and costs used for calculations are shown in Tables 2 and 3.

TABLE 1
Composition of molasses-based supplements containing different sources
and levels of total sulfur amino acids in undegraded intake protein.

| | | Concentration in Supplement, % as Fed | | | | | | |
|---|---|---|---|---|---|---|---|---|
| Bypass | | | | | Corn | | Supplement | Supplement |
| TSAA | | MOL- | | Ground | gluten | | Offered^{e} | Cost^{f} |
| g/d^{a} | Source | UREA^{b} | MOL^{c} | corn | meal | Alimet ®^{d} | lb/d | $/100 lb |
| .98 | Corn | 56.67 | 25.33 | 18.00 | .00 | .00 | 6.00 | 5.29 |
| 2.98 | CGM | 45.95 | 35.81 | 12.38 | 5.86 | .00 | 5.92 | 5.87 |
| 4.98 | CGM | 34.87 | 46.67 | 6.62 | 11.85 | .00 | 5.85 | 6.47 |
| 6.98 | CGM | 23.53 | 57.79 | .69 | 17.99 | .00 | 5.78 | 7.07 |
| 2.98 | Alimet ® | 56.52 | 25.27 | 18.00 | 0.00 | .20808 | 6.01 | 5.60 |
| 4.98 | Alimet ® | 56.37 | 25.21 | 18.00 | 0.00 | .41705 | 6.03 | 5.90 |
| 6.98 | Alimet ® | 56.22 | 25.15 | 18.00 | 0.00 | .62558 | 6.04 | 6.20 |
| 8.98 | Alimet ® | 56.07 | 25.10 | 18.00 | 0.00 | .83440 | 6.05 | 6.50 |

^{a}Calculated methionine and cystine (TSAA) contained in undegraded intake protein (UIP) based on values in references. Calculated bypass TSAA in UIP based ingredient analyses of crude protein (Table 2), insitu estimates of UIP (Table 3), and TSAA in UIP (Table 3) was 0.89, 2.73, 4.23, and 6.12 gm/d for corn and corn gluten meal treatments. Calculated bypass TSAA for Alimet ® is based upon a 40% bypass rate.
^{b}Molasses-Urea USSC product 502, fortified with .7% P, trace minerals and vitamins A, E, and D.
^{c}Molasses USSC product 701, fortified with .7% P, trace minerals and vitamins A, E, and D.
^{d}Alimet ®, Novus International, contains 88% methionine hydroxy analog (HMBA), 40% calculated to be absorbed as HMBA, $150./100 lb used to calculate supplement cost.
^{e}Supplement fed at level shown provided 3.28 lb total digestible nutrients (TDN)/d.
^{f}Cost calculated using ingredient costs in Table 2 and footnote d above.

TABLE 2
Analyzed composition of feeds used in molasses-based
supplements and bermudagrass hay.

| | | | | Corn | |
|---|---|---|---|---|---|
| | MOL- | | Corn, | gluten | Bermuda- |
| Item | UREA^{a} | MOL^{b} | ground | meal | grass hay |
| Number of samples | 4 | 4 | 4 | 4 | 4 |
| Dry matter, % | 73.2 | 74.4 | 86.8 | 91.3 | 89.7 |
| Crude protein, % DM | 23.8 | 11.6 | 8.5 | 63.8 | 11.4 |
| Soluble protein, | — | — | 19.8 | 5.5 | 22.8 |
| % DM | | | | | |
| Neutral detergent | — | — | 7.9 | 6.0 | 75.5 |
| fiber, % DM | | | | | |
| NSC, % DM^{c} | — | — | 78.1 | 24.4 | 4.0 |
| TDN, % DM | 66.0 | 70.0 | 88.5 | 86.0 | 54.5 |
| Total sugar as | 60.1 | 62.9 | — | — | — |
| invert, % DM | | | | | |
| Glucose, % DM | 1.91 | 2.28 | — | — | — |
| Fructose, % DM | 6.15 | 6.18 | — | — | — |
| Sucrose, % DM | 44.9 | 45.3 | | — | — |
| Calcium, % DM | 1.04 | 1.11 | 0.03 | 0.16 | 0.47 |
| Phosphorus, % DM | 0.95 | 0.88 | 0.34 | 0.50 | 0.31 |
| Magnesium, % DM | 0.46 | 0.50 | 0.12 | 0.06 | 0.21 |
| Potassium, % DM | 5.76 | 6.17 | 0.39 | 0.20 | 1.74 |
| Sodium, % DM | 0.18 | 0.19 | 0.01 | 0.06 | 0.05 |
| Sulfur, % DM | 0.978 | 1.024 | 0.083 | 0.930 | 0.123 |
| Iron, ppm in DM | 483 | 487 | 34 | 104 | 140 |
| Zinc, ppm in DM | 158 | 117 | 23 | 41 | 22 |
| Copper, ppm in DM | 70.6 | 75.4 | 2.5 | 3.5 | 6.0 |
| Manganese, ppm | 66.4 | 56.9 | 5.3 | 22.8 | 72.0 |
| in DM | | | | | |
| Cost of feed, $/100 lb | 5.40 | 4.20 | 6.50 | 18.50 | 3.00 |
| as fed | | | | | |

^{a}Molasses-Urea USSC product 502, fortified with .7% P, trace minerals and vitamins A, E, and D.
^{b}Molasses USSC product 701, fortified with .7% P, trace minerals and vitamins A, E, and D.
^{c}Non-structural (i.e., non-fiber) carbohydrates.

TABLE 3
In-situ undegraded intake protein (UIP) estimates and
amino acid concentrations of in-situ residues.

| | | | Corn | | | |
|---|---|---|---|---|---|---|
| | Ground corn | | gluten meal | | | |
| | | % | | % | Bermudagrass | |
| Item | % CP | EAA^{c} | % CP | EAA^{c} | % CP | % EAA^{c} |
| Feed crude protein, | 8.50 | — | 63.80 | — | 11.40 | — |
| % DM | | | | | | |
| Insitu crude protein, | 11.13 | — | 79.58 | — | 6.56 | — |
| % DM | | | | | | |
| Insitu UIP, % | 54.16 | — | 50.39 | — | 15.03 | — |
| initial protein | | | | | | |
| Methionine | 2.34 | 5.52 | 2.51 | 5.76 | 2.44 | 5.05 |
| Cysteine | 2.25 | — | 1.87 | — | 1.52 | — |
| TSAA^{b} | 4.59 | — | 4.39 | — | 3.96 | — |
| Lysine | 2.16 | 5.10 | 1.77 | 4.06 | 4.88 | 10.09 |
| Histidine | 2.61 | 6.16 | 2.07 | 4.75 | 1.52 | 3.15 |
| Isoleucine | 3.68 | 8.70 | 3.67 | 8.41 | 5.49 | 11.36 |
| Leucine | 14.65 | 34.61 | 16.34 | 37.45 | 10.21 | 21.14 |
| Arginine | 3.14 | 7.43 | 3.17 | 7.26 | 4.88 | 10.09 |
| Phenylalanine | 5.48 | 12.95 | 6.01 | 13.77 | 6.10 | 12.62 |
| Threonine | 3.41 | 8.07 | 3.18 | 7.29 | 5.03 | 10.41 |
| Tryptophan | 0.45 | 1.06 | 0.59 | 1.35 | 1.52 | 3.15 |
| Valine | 4.40 | 10.40 | 4.31 | 9.88 | 6.25 | 12.93 |
| Hydroxyprolin | 0.45 | — | 0.01 | — | 0.61 | — |
| Hydroxilysine | 0.00 | — | 0.00 | — | 0.00 | — |
| Taurine | 1.80 | — | 0.04 | — | 0.76 | — |
| Lanthionine | 0.00 | — | 0.03 | — | 0.00 | — |
| Ornithine | 0.09 | — | 0.08 | — | 0.15 | — |
| Tyrosine | 3.86 | — | 5.05 | — | 3.66 | — |
| Serine | 4.13 | — | 3.97 | — | 4.42 | — |
| Aspartic acid | 5.66 | — | 5.78 | — | 10.37 | — |
| Glutamic acid | 19.23 | — | 19.46 | — | 11.89 | — |
| Proline | 9.43 | — | 9.01 | — | 5.49 | — |
| Glycine | 3.23 | — | 2.76 | — | 5.64 | — |
| Alanine | 7.73 | — | 8.34 | — | 7.01 | — |

^{a}Crude protein in in-situ residue, analyzed by Novus, used to calculate % AA as % CP
^{b}Total sulfur amino acids (Met + Cys).
^{c}Each essential amino acid divided by total essential amino acids (Met, Lys, His, Ile, Leu, Arg, Phe, Thr, Trp, Val).

This experiment was conducted at the Santa Fe Beef Unit located near Gainesville. Thirty-two pens (4 head/pen) of Angus-Brahman crossbred cattle (9-12 months of age) were assigned randomly within sex and breed type. The calves averaged 610 lb and calves were in excellent body condition (BCS 5.8) at the start of the trial. Each pen was assigned two steers and two heifers. These 32 pens were randomly assigned to the 8 treatments. Each pen was a two-acre pasture and most grass was grazed off at the start of the trial but warm weather allowed some grass growth during February and March. Molasses slurries were fed on two days each week (equivalent amount for 3 or 4 days at each feeding) at the equivalent daily rate. Molasses slurries were hand mixed in each pen and fed in open troughs. Alimet® was pre-weighed in the laboratory and mixed in the supplement of the assigned pen at each feeding. Bermuda grass hay was offered ad libitum in hay rings outside (no shelter) and molasses slurries were limit fed during the 112-day trial (December thru April). Hay not consumed was collected, weighed, sampled (dry matter analyzed), and removed from the pens every 6 to 8 weeks during the trial. Water and a mineral-vitamin supplement were offered ad libitum.

Supplement consumption, hay consumption, full weight, shrunk weight (beginning and end only), height, body condition, and blood urea (2 calves/pen) were measured at 28-day intervals during the 112-day trial. Supplement ingredients were sampled twice monthly and each hay bale was weighed and sampled prior to feeding. Supplement ingredients were analyzed for dry matter, organic matter, crude protein, bypass protein, amino acids, and minerals. Hay samples were analyzed for dry matter, organic matter, crude protein, amino acids, in vitro organic matter digestibility, neutral detergent fiber, and minerals.

### Results and Discussion

Cattle were limit-fed the molasses slurry supplements (Table 1) and all supplements offered were consumed after the second week for all treatments except the high level (22.7 g/day) of Alimet®. Supplement consumption averaged 0.5 lb/day lower during the trial for the high level of Alimet® (see Table 4). Feeding up to 11 g/day of Alimet® resulted in only two pens with supplement refusal after the first feeding which was similar to corn gluten meal (see Table 5). Feeding 17 g/day of Alimet® resulted in only one instance of supplement refusal after the first week. Feeding 22.7 g/day of Alimet®, however, resulted in supplement refusals for several weeks in two of the four pens offered the supplement. Supplement dry matter consumption by 28-day period (see Table 6) shows the lower supplement consumption for the first 84 days of the 112-day trial.

TABLE 4
Effect of source and level of supplemental bypass total sulfur amino acids (TSAA) on performance of growing
cattle fed bermudagrass hay diets supplemented with molasses based supplements.

| | | Supplemental bypass TSAA | | | Supplemental bypass TSAA provided | | | | |
|---|---|---|---|---|---|---|---|---|---|
| | | provided by corn gluten meal | | | by Alimet (methionine hydroxy analog) | | | | |
| Item | Control | bypass/d | bypass/d | bypass/d | bypass/d | bypass/d | bypass/d | bypass/d | SE |
| Number of pens | 4 | 4 | 4 | 4 | 4 | 4 | 4 | 4 | — |
| Initial shrunk weight, (12/13/00) | 610 | 607 | 603 | 606 | 612 | 614 | 600 | 612 | 7.1 |
| Daily gain, lb/d | | | | | | | | | |
| 0-28 days (full wt) | 1.68 | 1.82 | 2.14 | 2.29 | 1.58 | 1.88 | 1.77 | 1.24 | .22 |
| 28-56 days (full wt) | .88 | 1.59 | 1.65 | 1.67 | 1.92 | 1.46 | 1.68 | 1.49 | .14 |
| 56-84 days (full wt) | 1.32 | 1.03 | .95 | .95 | 1.09 | .99 | 1.40 | .93 | .14 |
| 84-112 days (full wt) | 1.95 | 2.43 | 2.08 | 2.29 | 1.90 | 1.82 | 1.98 | 2.48 | .19 |
| 0-112 days (full wt) | 1.44 | 1.70 | 1.69 | 1.79 | 1.61 | 1.53 | 1.69 | 1.52 | .08 |
| 1-113 days (shrunk wt) | 1.18^{a} | 1.41^{bc} | 1.43^{bc} | 1.49^{c} | 1.35^{bc} | 1.31^{ab} | 1.44^{bc} | 1.31^{ab} | .06 |
| Added gain, lb/d | — | .23 | .25 | .31 | .17 | .13 | .26 | .13 | — |
| Initial height, in. (12/13/00) | 47.3 | 47.5 | 47.4 | 47.8 | 47.7 | 47.5 | 47.2 | 47.4 | .27 |
| Height change, in. (0-112 days) | 2.70 | 2.96 | 3.16 | 2.79 | 2.73 | 2.45 | 2.80 | 2.79 | .31 |
| Initial BCS (12/12/00) | 5.87 | 5.87 | 5.81 | 5.74 | 5.88 | 5.78 | 5.94 | 5.89 | .07 |
| BCS change (0-112 days) | +.18 | +.18 | +.09 | +.36 | +.14 | +.09 | +.18 | +.08 | .13 |
| Feed consumed, lb/d DM | | | | | | | | | |
| Hay | 8.85^{a} | 9.89^{ab} | 10.34^{b} | 9.97^{ab} | 11.23^{b} | 10.49^{b} | 11.04^{b} | 10.49^{b} | .46 |
| Supplement | 4.52^{a} | 4.51^{a} | 4.43^{a} | 4.43^{a} | 4.51^{a} | 4.51^{a} | 4.45^{a} | 4.08^{b} | .06 |
| Total | 13.36^{a} | 14.40^{ab} | 14.77^{b} | 14.40^{ab} | 15.74^{b} | 14.99^{b} | 15.49^{b} | 14.57^{ab} | .48 |
| TDN consumed, lb/d | 8.00^{a} | 8.54^{ab} | 8.72^{ab} | 8.58^{ab} | 9.21^{b} | 8.84^{b} | 9.13^{b} | 8.56^{ab} | .27 |
| Feed efficiency | | | | | | | | | |
| Gain/feed DM | .089^{ac} | .099^{ab} | .098^{ab} | .104^{b} | .087^{c} | .088^{c} | .094^{a} | .091^{a} | .004 |
| Gain/TDN | .149^{ad} | .167^{bd} | .167^{bd} | .175^{b} | .149^{acd} | .149^{acd} | .159^{ad} | .154^{d} | .006 |
| Hay offered, lb/d as fed | 10.65^{a} | 12.18^{b} | 12.52^{b} | 12.03^{a} | 13.25^{b} | 12.48^{b} | 13.23^{b} | 12.46^{b} | .51 |
| Supplement offered, lb/d as fed | 5.95^{a} | 5.91^{a} | 5.79^{a} | 5.75^{a} | 5.94^{a} | 5.95^{a} | 5.87^{a} | 5.33^{b} | .09 |
| Supplement cost, cents/d | 31.5^{a} | 34.7^{b} | 37.5^{c} | 40.7^{d} | 33.3^{b} | 35.1^{b} | 36.4^{bc} | 34.8^{b} | .58 |
| Added supplement cost, cents/d | — | 3.2 | 6.0 | 9.2 | 1.8 | 3.6 | 4.9 | 3.3 | — |
| Cost added gain, cents/added lb | — | 13.9 | 24.0 | 29.7 | 10.6 | 27.7 | 18.8 | 25.4 | — |
| Total feed cost, cents/d | 63.4^{a} | 71.2^{b} | 75.0^{bc} | 76.8^{c} | 73.0^{b} | 72.5^{b} | 76.1^{b} | 72.0^{b} | 1.8 |
| Total feed cost, cents/lb gain | 53.8 | 50.2 | 51.9 | 51.1 | 53.6 | 55.2 | 52.8 | 54.7 | 2.3 |

^{abcd}Means with different superscripts differ (P < .05).

Liquid Supplement Refusal by Pen (4/treatment) Fed
Alimet in a Molasses Supplement^{a}

| TRT | | | | |
|---|---|---|---|---|
| Feed | Alimet - | Alimet - | Alimet - | Alimet - |
| Date | 5.7 g/d | 11.4 g/d | 17 g/d | 22.7 g/d |

| PEN | 1 | 9 | 11 | E | 8 | 20 | A | D | 5 | 12 | 25 | G | 4 | 14 | 26 | F |
|---|---|---|---|---|---|---|---|---|---|---|---|---|---|---|---|---|
| 12.18 | 25 | | | | 35 | | | | | 10 | 45 | | 20 | 35 | 20 | |
| 12.22 | | | | | | | | | 15 | 10 | 20 | | 25 | 20 | | |
| 12.26 | | | | | | | | | | | | | 30 | 60 | | |
| 12.29 | | | | | | | | | | | | | 35 | 55 | | |
| 1.02 | | | | | | | | | | 25 | | | | 75 | | |
| 1.05 | | | | | | | | | | | | | 30 | 45 | | |
| 1.08 | | | | | | | | | | | | | | | 30 | |
| 1.12 | | | | | | | | | | | | | 30 | | 45 | |
| 1.16 | | | | | | | | | | | | | 25 | | | |
| 1.19 | | | | | | | | | | | | | | | | |
| 1.22 | | | | | | | | | | | | | 30 | 25 | | 20 |
| 1.25 | | | | | | | | | | | | | | 30 | | |
| 1.29 | | | | | | | | | | | | | 30 | | | |
| 2.01 | | | | | | | | | | | | | | | | |
| 2.05 | | | | | | | | | | | | | 20 | | | |
| 2.08 | | | | | | | | | | | | | 30 | | | |
| 2.12 | | | | | | | | | | | | | | 30 | | |
| 2.15 | | | | | | | | | | | | | | | | |
| 2.19 | | | | | | | | | | | | | 40 | 35 | | |
| 2.22 | | | | | | | | | | | | | 20 | | | |
| 2.26 | | | | | | | | | | | | | 20 | | | |
| 3.01 | | | | | | | | | | | | | 20 | 45 | | 35 |
| 3.05 | | | | | | | | | | | | | 35 | 35 | | |
| 3.08 | | | | | | | | | | | | | 20 | | | 20 |
| 3.12 | | | | | | | | | | | | | | | | |
| 3.15 | | | | | | | | | | | | | | | | |
| 3.19 | | | | | | | | | | | | | | | | |
| 3.23 | | | | | | | | | | | | | | | | |
| 3.27 | | | | | | | | | | | | | | | | |
| 3.30 | | | | | | | | | | | | | | | | |
| 4.02 | | | | | | | | | | | | | | | | |
| 4.04 | | | | | | | | | | | | | | | | |

^{a}Numbers are pounds of molasses based supplement remaining from last feeding on each feeding
date. Empty spaces indicate no supplement was left in the feeder.

TABLE 6
Supplement and hay dry matter (DM) offered by period for 112-day trial.

| | | Supplemental bypass | | | Supplemental bypass TSAA | | | |
|---|---|---|---|---|---|---|---|---|
| | | TSAA provided by | | | from Alimet | | | |
| | | corn gluten meal | | | (methionine hydroxy analog) | | | |
| | | 2 gm | 4 gm | 6 gm | 2 gm | 4 gm | 6 gm | 8 gm |
| Period Feed | | bypass/ | bypass/ | bypass/ | bypass/ | bypass/ | bypass/ | bypass/ |
| offered | Control | day | day | day | day | day | day | day |
| 0-28 days of trial | | | | | | | | |
| Molasses | 1.15 | 1.61 | 2.02 | 2.50 | 1.14 | 1.13 | 1.10 | .98 |
| Molasses-urea | 2.56 | 2.03 | 1.48 | 1.01 | 2.54 | 2.53 | 2.37 | 2.18 |
| Corn | .94 | .67 | .34 | .05 | .96 | .95 | .92 | .82 |
| Corn gluten meal | 0 | .34 | .61 | .97 | 0 | 0 | 0 | 0 |
| Alimet | 0 | 0 | 0 | 0 | .01 | .02 | .03 | .04 |
| Total supplement | 4.65 | 4.65 | 4.45 | 4.53 | 4.65 | 4.63 | 4.42 | 4.02 |
| Hay offered | 14.59 | 15.85 | 16.17 | 16.08 | 16.23 | 15.69 | 15.39 | 16.64 |
| Total DM offered | 19.24 | 20.50 | 20.62 | 20.61 | 20.88 | 20.32 | 19.81 | 20.66 |
| 28-56 days of trial | | | | | | | | |
| Molasses | 1.07 | 1.50 | 1.96 | 2.37 | 1.07 | 1.07 | 1.07 | .97 |
| Molasses-urea | 2.39 | 1.92 | 1.43 | .95 | 2.39 | 2.39 | 2.39 | 2.16 |
| Corn | .90 | .63 | .33 | .04 | .90 | .90 | .90 | .81 |
| Corn gluten meal | 0 | .31 | .59 | .94 | 0 | 0 | 0 | 0 |
| Alimet | 0 | 0 | 0 | 0 | .01 | .02 | .03 | .04 |
| Total supplement | 4.36 | 4.36 | 4.31 | 4.30 | 4.37 | 4.38 | 4.39 | 3.98 |
| Hay offered | 9.49 | 10.44 | 11.83 | 7.86 | 11.20 | 10.88 | 12.04 | 10.47 |
| Total DM offered | 13.85 | 14.80 | 16.14 | 12.16 | 15.57 | 15.26 | 16.43 | 14.45 |
| 56-84 days of trial | | | | | | | | |
| Molasses | 1.11 | 1.56 | 2.04 | 2.46 | 1.11 | 1.11 | 1.11 | .97 |
| Molasses-urea | 2.48 | 1.98 | 1.49 | .99 | 2.48 | 2.48 | 2.48 | 2.16 |
| Corn | .93 | .65 | .34 | .05 | .93 | .93 | .93 | .82 |
| Corn gluten meal | 0 | .33 | .62 | .95 | 0 | 0 | 0 | 0 |
| Alimet | 0 | 0 | 0 | 0 | .01 | .02 | .03 | .04 |
| Total supplement | 4.52 | 4.52 | 4.49 | 4.45 | 4.53 | 4.54 | 4.55 | 3.99 |
| Hay offered | 8.33 | 7.74 | 8.38 | 10.54 | 10.32 | 8.82 | 9.14 | 10.01 |
| Total DM offered | 12.85 | 12.26 | 12.77 | 14.99 | 14.85 | 13.36 | 13.69 | 14 |
| 84-112 days of trial | | | | | | | | |
| Molasses | 1.11 | 1.56 | 2.04 | 2.46 | 1.11 | 1.11 | 1.11 | 1.09 |
| Molasses-urea | 2.48 | 1.98 | 1.49 | .99 | 2.48 | 2.48 | 2.48 | 2.44 |
| Corn | .93 | .65 | .34 | .05 | .93 | .93 | .93 | .92 |
| Corn gluten meal | 0 | .33 | .62 | .96 | 0 | 0 | 0 | 0 |
| Alimet | 0 | 0 | 0 | 0 | .01 | .02 | .03 | .04 |
| Total supplement | 4.56 | 4.56 | 4.52 | 4.49 | 4.53 | 4.54 | 4.55 | 4.49 |
| Hay offered | 7.24 | 10.25 | 9.69 | 9.80 | 9.76 | 10.80 | 12.26 | 9.52 |
| Total DM offered | 11.80 | 14.81 | 14.21 | 14.29 | 14.29 | 15.34 | 16.81 | 14.01 |
| 0-112 days of trial | | | | | | | | |
| Molasses | 1.11 | 1.56 | 2.01 | 2.45 | 1.11 | 1.11 | 1.10 | 1.00 |
| Molasses-urea | 2.48 | 1.98 | 1.47 | .99 | 2.47 | 2.47 | 2.43 | 2.23 |
| Corn | .93 | .65 | .34 | .05 | .93 | .93 | .92 | .84 |
| Corn gluten meal | 0 | .32 | .61 | .95 | 0 | 0 | 0 | 0 |
| Alimet | 0 | 0 | 0 | 0 | .01 | .02 | .03 | .04 |
| Total supplement | 4.52 | 4.51 | 4.43 | 4.44 | 4.52 | 4.53 | 4.48 | 4.11 |
| Hay offered | 9.92 | 11.07 | 11.52 | 11.07 | 11.88 | 11.55 | 12.21 | 11.66 |
| Total DM offered | 14.44 | 15.58 | 15.95 | 15.51 | 16.4 | 16.08 | 16.69 | 15.77 |

^{a}Dry matter intakes calculated using estimated dry matter concentrations for ingredients.

During the first 28 days, cattle on all treatments except the highest level (22.7 g/d) of Alimet® gained well (1.58 to 2.29 lb/d). During the 112-day trial, cattle fed corn gluten meal (CGM) at 2 gm/day, 4 gm/day, or 6 gm/day of bypass TSAA had similar performances (1.43 to 1.49 lb/day shrunk wt gain) and gains were 0.23 to 0.31 lb/day above cattle fed the control supplement. Feeding Alimet® to provide 2, 4, or 6 gm/day of bypass TSAA resulted in 0.13 to 0.26 lb/day higher gains than cattle fed the control supplement. Treatment differences on height or body condition change were not found.

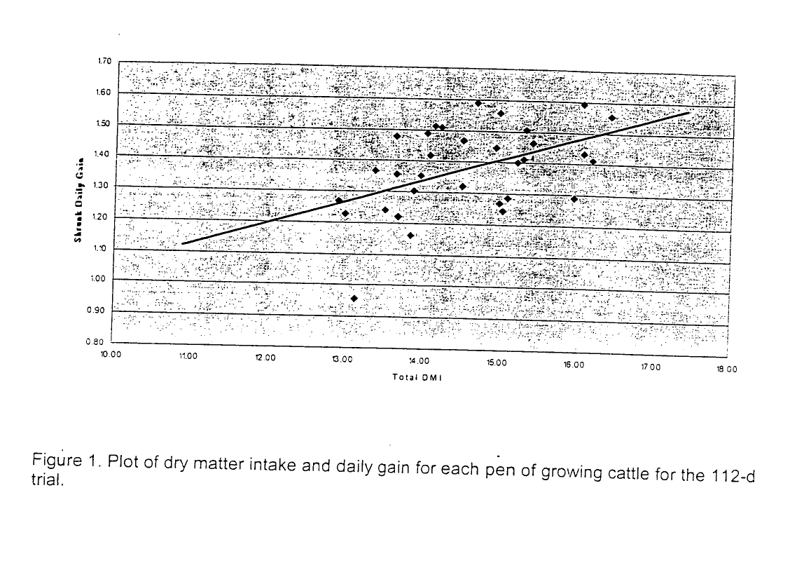

Estimated dry matter intake showed a trend for higher hay consumption for treatments fed both bypass TSAA sources. Gain for each unit of feed (DM) was within a narrow range (0.87 to 0.104 lb gain/lb feed DM). A plot of dry matter intake and gains (FIG. 1) shows a trend for higher gains with higher DM intake but considerable variation exists.

Daily supplement cost increased when corn gluten meal and Alimet® were fed. The 6 gm/day bypass TSAA gave the highest gains for both sources and the cost of added gain was 29.7 cents/lb gain for CGM but only 19.7 cents/lb gain for Alimet®. Alimet® had a 10 cents/lb added gain cost advantage compared to CGM and gain was well below the value of added gain at current calf prices. The cost of added gain was lower for Alimet® than other bypass TSAA sources evaluated in previous trials evaluating rumen protected encapsulated methionine sources. Feeding 17 gm of Alimet® daily (6 gm/day TSAA) cost under 6 cents and produced over 20 cents of increased gain at current calf prices, over a $3 return for each dollar spent.

In view of the above, it will be seen that the several objects of the invention are achieved and other advantageous results attained.

As various changes could be made in the above feed rations and methods without departing from the scope of the invention, it is intended that all matter contained in the above description and shown in the accompanying drawing shall be interpreted as illustrative and not in a limiting sense.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

We claim:

1. A method of improving weight gain of a growing ruminant comprising:
providing the ruminant one or more feed ingredients; and
providing the ruminant an α-amino acid analog selected from the group consisting of 2-hydroxy-4-(methylthio)butanoic acid, and salts, esters, amides, ethers, diesters, ester/ethers, oligomers, metal chelates, and anion salts thereof; and the salt, ester, amide, ether, oligomer, metal chelate, and anion salt analogs of methionine;
wherein the assortment and composition of said feed ingredients are such that the amounts thereof which can be consumed by the growing ruminant in one day can collectively satisfy the ruminant's daily nutrient requirements, and exceed its daily maintenance energy requirements; provided that such assortment and composition may not necessarily satisfy the growing ruminant's methionine requirements; and
wherein the salts of 2-hydroxy-4-(methylthio)butanoic acid are selected from the group consisting of ammonium, magnesium, lithium, sodium, potassium, and zinc.

2. The method of claim 1, wherein the α-amino acid analog is selected from the group consisting of methyl, ethyl, n-propyl, isopropyl, n-butyl, sec-butyl, isobutyl, tertiary butyl, n-pentyl, isopentyl, -hexyl, and isohexyl esters of 2-hydroxy-4-(methylthio)butanoic acid.

3. The method of claim 1, wherein the α-amino acid analog is selected from the group consisting of methylamide, dimethylamide, ethylmethylamide, butylamide, dibutylamide, butylmethylamide, alkyl ester of N-acyl methioninates, and alkyl N-acetyl methioninate amides of 2-hydroxy-4-(methylthio)butanoic acid.

4. The method of claim 1, wherein the α-amino acid analog is selected from the group consisting of methyl, ethyl, n-propyl, isopropyl, butyl, n-butyl, sec-butyl, isobutyl, tertiary butyl, pentyl, n-pentyl, isopentyl, hexyl, and isohexyl ethers of 2-hydroxy-4-(methylthio)butanoic acid.

5. The method of claim 1, wherein the ester group of the ester/ether of 2-hydroxy-4-(methylthio)butanoic acid is selected from the group consisting of methyl, ethyl, n-propyl, isopropyl, n-butyl, sec-butyl, isobutyl, tertiary butyl, n-pentyl, isopentyl, n-hexyl, and isohexyl esters and the ether group of the ester/ether of 2-hydroxy-4-(methylthio)butanoic acid is selected from the group consisting of methyl, ethyl, n-propyl, isopropyl, n-butyl, sec-butyl, isobutyl, tertiary butyl, n-pentyl, isopentyl, n-hexyl, and isohexyl ethers.

6. The method of claim 1, wherein the α-amino acid analog is selected from the group consisting of zinc, copper, cobalt, manganese, calcium, iron, or magnesium chelates of 2-hydroxy-4-(methylthio)butanoic acid.

7. The method of claim 1, wherein the metal chelate of 2-hydroxy-4-(methylthio)butanoic acid comprises the zinc chelate.

8. The method of claim 8, wherein the molar ratio of 2-hydroxy-4-(methylthio)butanoic acid to zinc is between about 1.5:1 to about 2.5:1.

9. The method of claim 1, wherein the anion salt of 2-hydroxy-4-(methylthio)butanoic acid is selected from the group consisting of zinc, copper, cobalt, manganese, calcium, iron, or magnesium anion salts.

10. The method of claim 9, wherein the anion salt of 2-hydroxy-4-(methylthio)butanoic acid comprises the zinc anion salt.

11. The method of claim 10, wherein the ratio of 2-hydroxy-4-(methylthio)butanoic acid to zinc is between about 0.5:1 to about 1.5:1.

12. The method of claim 1, wherein the α-amino acid analog comprises the isopropyl ester of 2-hydroxy-4-(methylthio)butyric acid.

13. The method of claim 1, wherein the α-amino acid analog comprises 2-hydroxy-4-(methylthio)butyric acid.

14. The method of claim 1, wherein the α-amino acid analog is selected from the group consisting of ammonium, magnesium, calcium, lithium, sodium, potassium, and zinc salts of methionine.

15. The method of claim 1, wherein the α-amino acid analog is selected from the group consisting of methyl, ethyl, n-propyl, isopropyl, n-butyl, sec-butyl, isobutyl, tertiary butyl, n-pentyl, isopentyl, -hexyl, and isohexyl esters of methionine.

16. The method of claim 1, wherein the α-amino acid analog is selected from the group consisting of methylamide, dimethylamide, ethylmethylamide, butylamide, dibutylamide, butylmethylamide, alkyl ester of N-acyl methioninates, and alkyl N-acetyl methioninates of methionine.

17. The method of claim 1, wherein the α-amino acid analog is selected from the group consisting of methyl, ethyl, n-propyl, isopropyl, n-butyl, sec-butyl, isobutyl, tertiary butyl, n-pentyl, isopentyl, -hexyl and isohexyl ethers of methionine.

18. The method of claim 1, wherein the α-amino acid analog is selected from the group consisting of zinc, copper, cobalt, manganese, calcium, iron, or magnesium chelates of methionine.

19. The method of claim 1, wherein the metal chelate of methioinine comprises the zinc chelate of methionine.

20. The method of claim 19, wherein the molar ratio of methionine to zinc is between about 1.5:1 to about 2.5:1.

21. The method of claim 1, wherein the α-amino acid analog is selected from the group consisting of zinc, copper, cobalt, manganese, calcium, iron, or magnesium anion salts of methionine.

22. The method of claim 21, wherein the anion salt of methionine comprises the zinc anion salt of methionine.

23. The method of claim 22, wherein the ratio of methionine to zinc is between about 0.5:1 to about 1.5:1.

24. The method of claim 1, wherein the analog of methionine is selected from the group consisting of the isopropyl ester, and tertiary butyl ester of methionine.

25. The method of claim 1, wherein the feed ingredients comprise fat.

26. The method of claim 1, further comprising a lysine analog.

27. The method of claim 26, wherein the lysine analog is selected from a the group consisting of lysine salts, amides, esters, ethers and oligomers.

28. The method of claim 27, wherein the lysine analog and the methionine analog are provided in a manner such that the ratio of the lysine analog to methionine analog available for absorption by the ruminant is between about 1:1 to about 5:1.

29. The method of claim 1, wherein the feed ingredients comprise urea.

30. The method of claim 1, wherein the feed ingredients comprise corn.

31. The method of claim 1, wherein the feed ingredients comprise forage.

32. The method of claim 31, wherein the forage is selected from the group consisting of grass, alfalfa, hay, haylage, or silage.

33. The method of claim 1, wherein the combination of feed ingredient and an α-amino acid analog are made available to the ruminant in the late summer through winter seasons.

34. The method of claim 1, wherein the growing ruminant is selected from growing beef or dairy cattle.

35. The method of claim 1, wherein the growing ruminant weighs less than 1000 pounds.

36. The method of claim 1, wherein the growing ruminant weighs between about 200 to about 1000 pounds.

37. The method of claim 1, wherein the growing ruminant weighs between about 400 to about 1000 pounds.

38. The method of claim 1, wherein the growing ruminant weighs between about 400 to about 800 pounds.

39. The method of claim 1, wherein the growing ruminant is a creep-fed ruminant weighing between about 200 to about 400 pounds.

40. The method of claim 1, wherein the α-amino acid analog is provided to the ruminant in an amount such that the ruminant consumes between about 0.1 and 50 grams of the α-amino acid analog per day.

41. The method of claim 1, wherein the α-amino acid analog is provided to the ruminant in an amount such that the ruminant consumes between about 1 and 25 grams of the α-amino acid analog per day.

42. The method of claim 1, wherein the α-amino acid analog is provided to the ruminant in an amount such that the ruminant consumes between about 5 and 20 grams of the α-amino acid analog per day.

43. The method of claim 1, wherein the α-amino acid analog is provided to the ruminant in an amount such that the ruminant consumes between about 10 and 18 grams of the α-amino acid analog per day.

44. The method of claim 1, wherein said feed ingredient comprises at least 50% forage ingredients.

45. The method of claim 1, wherein said feed ingredient comprises at least 65% forage ingredients.

46. The method of claim 1, wherein said feed ingredient comprises at least 80% forage ingredients.

47. The method of claim 1, wherein said feed ingredient comprises at least 80% forage ingredients.

48. The method of claim 1, wherein said feed ingredient comprises at least 85% forage ingredients.

49. The method of claim 1, wherein said feed ingredient further comprises molasses.

50. The method of claim 49, wherein at least 0.5 pounds per day of molasses is provided to the ruminant.

51. The method of claim 49, wherein 1 to 7 pounds per day of molasses is provided to the ruminant.

52. The method of claim 49, wherein the ratio of forage to molasses feed ingredients provided to the growing ruminant is between about 20:1 to about 1.5:1.

53. The method of claim 49, wherein the ratio of forage to molasses feed ingredients provided to the growing ruminant is between about 10:1 to about 2:1.

54. The method of claim 49, wherein the ratio of forage to molasses feed ingredients provided to the growing ruminant is between about 7:1 to about 2.5:1.

55. The method of claim 1, wherein the growing ruminant is a ruminant selected from beef or dairy cattle.

56. The method of claim 1, wherein the ration is top-dressed with the α-amino acid analog.

57. The method of claim 1, wherein the ration is top-dressed with the α-amino acid analog.

58. The method of claim 1, wherein the feed ration is formulated to provide between about 4 and about 30 pounds of feed ingredients and between about 0.1 to about 50 grams of α-amino acid analog per day.

59. The method of claim 58, wherein the feed ration is provided to a growing ruminant weighing between about 400 and about 1000 pounds.

60. The method of claim 58, wherein the feed ration is formulated to provide between about 8 and about 24 pounds of feed ingredients per day.

61. The method of claim 60, wherein the feed ration is provided to a growing ruminant weighing between about 400 and about 800 pounds.

62. The method of claim 58, wherein the feed ration is formulated to provide between about 4 and about 12 pounds of feed ingredients per day.

63. The method of claim 62, wherein the feed ration is provided to a growing ruminant weighing between about 200 and about 400 pounds.

64. The method of claim 1, wherein the feed ingredients collectively satisfy the nutritional requirements for methionine.

65. The method of claim 1, wherein the feed ingredients collectively are deficient in satisfying the nutritional requirements for methionine.

66. The method of claim 1, further comprising determining the energy maintenance requirements of the growing ruminant.

67. A method of improving weight gain of a growing ruminant comprising:
providing the ruminant one or more feed ingredients, and
providing the ruminant an α-amino acid analog selected from the group consisting of the calcium salt of 2-hydroxy-4-(methylthio)butanoic acid.
wherein the combination of feed ingredients comprise at least 80% forage, and
wherein the assortment and composition of said feed ingredients are such that the amounts thereof which can be consumed by the growing ruminant in one day can collectively satisfy the ruminant's daily nutrient requirements, and exceed its daily maintenance energy requirements; provided that such assortment and composition may not necessarily satisfy the growing ruminant's methionine requirements.

68. The method of claim 67, wherein the calcium salt of 2-hydroxy-4-(methylthio)butanoic acid is provided to the ruminant in an amount such that the ruminant consumes between about 0.1 and 50 grams of the α-amino acid analog per day.

69. The method of claim 67, wherein the calcium salt of 2-hydroxy-4-(methylthio)butanoic acid is provided to the ruminant in an amount such that the ruminant consumes between about 1 and 25 grams of the α-amino acid analog per day.

70. The method of claim 67, wherein the growing ruminant weighs between about 400 to about 800 pounds.

71. The method of claim 67, wherein the growing ruminant is a creep-fed ruminant weighing between about 200 to about 400 pounds.

72. The method of claim 67, wherein said feed ingredient further comprises molasses.

73. The method of claim 72, wherein at least 0.5 pounds per day of molasses is provided to the ruminant.

74. The method of claim 67, wherein the ration is top-dressed with the calcium salt of 2-hydroxy-4-(methylthio)butanoic acid.

75. The method of claim 67, wherein the feed ration is formulated to provide between about 4 and about 30 pounds of feed ingredients and between about 0.1 to about 50 grams of calcium salt of 2-hydroxy-4-(methylthio)butanoic acid per day.

76. The method of claim 75, wherein the feed ration is provided to a growing ruminant weighing between about 400 and about 1000 pounds.

77. The method of claim 75, wherein the feed ration is formulated to provide between about 8 and about 24 pounds of feed ingredients per day.

78. The method of claim 77, wherein the feed ration is provided to a growing ruminant weighing between about 400 and about 800 pounds.

79. The method of claim 75, wherein the feed ration is formulated to provide between about 4 and about 12 pounds of feed ingredients per day.

80. The method of claim 79, wherein the feed ration is provided to a growing ruminant weighing between about 200 and about 400 pounds.

81. The method of claim 67, further comprising determining the energy maintenance requirements of the growing ruminant.

82. The method of claim 67, wherein the feed ingredients collectively satisfy the nutritional requirements for methionine.

83. The method of claim 67, wherein the feed ingredients collectively do not satisfy the nutritional requirements for methionine.

84. A process of formulating a feed ration for growing ruminants, the process comprising:
determining the nutritional requirements of the growing ruminant;
determining the maintenance energy requirements of the growing ruminant;
identifying one or more feed ingredients other than α-amino acid analogs and determining the nutritional content and energy contribution of each of said feed ingredients;
formulating a feed ration from the identified feed ingredients such that the amount of feed ration which can be consumed by the growing ruminant in one day can collectively satisfy the ruminant's daily nutrient requirements, and exceed its daily maintenance energy requirements; provided that such assortment and composition may not necessarily satisfy the growing ruminant's methionine requirements; and
additionally incorporating into the feed ration an α-amino acid analog without regard to its energy contribution to the feed ration.

85. The process of claim 84, wherein the α-amino acid analog is incorporated in the feed ration in an amount to provide between about 0.1 and 50 grams of the α-amino acid analog per day.

86. The process of claim 84, wherein the α-amino acid analog is incorporated in the feed ration in an amount to provide between about 1 and 25 grams of the α-amino acid analog per day.

87. The process of claim 84, wherein the α-amino acid analog is selected from the group consisting of 2-hydroxy-4-(methylthio)butanoic acid, and salts, esters, amides, ethers, diesters, ester/ethers, oligomers, metal chelates, and anion salts thereof.

88. The process of claim 84, wherein the feed ration comprises at least 50% forage ingredients.

89. The process of claim 84, wherein the feed ration comprises at least 80% forage ingredients.

90. The process of claim 84, wherein the α-amino acid analog comprises 2-hydroxy-4-(methylthio)butanoic acid.

91. The process of claim 84, wherein the α-amino acid analog comprises the isopropyl ester of 2-hydroxy-4-(methylthio)butanoic acid.

92. The process of claim 84, wherein the α-amino acid analog comprises the tertiary butyl ester of 2-hydroxy-4-(methylthio)butanoic acid.

93. The process of claim 84, wherein the feed ration satisfies the nutritional requirements for methionine.

94. The process of claim 84, wherein the feed ration does not satisfy the nutritional requirements for methionine.

95. A feed ration for growing ruminants comprising:
one or more feed ingredients and an α-amino acid analog;
wherein the feed ration comprises at least 50% forage;
wherein the α-amino acid analog is selected from the group consisting of 2-hydroxy-4-(methylthio)butanoic acid, and salts, esters, amides, ethers, diesters, ester/ethers, oligomers, metal chelates, and anion salts thereof; and the salt, ester, amide, ether, oligomer, metal chelate, and anion salt analogs of methionine; and
wherein the salts of 2-hydroxy-4-(methylthio)butanoic acid are selected from the group consisting of ammonium, magnesium, lithium, sodium, potassium, and zinc;
the feed ration being formulated such that an amount of feed ration that is capable of being consumed by a growing ruminant in a day satisfies the ruminant's daily nutritional requirements, and exceeds its daily maintenance energy requirements; provided that such assortment and composition may not necessarily satisfy the growing ruminant's methionine requirements.

96. The feed ration of claim 95, wherein the α-amino acid analog is selected from the group consisting of methyl, ethyl, n-propyl, isopropyl, n-butyl, sec-butyl, isobutyl, tertiary butyl, n-pentyl, isopentyl, -hexyl, and isohexyl esters of 2-hydroxy-4-(methylthio)butanoic acid.

97. The feed ration of claim 95, wherein the α-amino acid analog is selected from the group consisting of methylamide, dimethylamide, ethylmethylamide, butylamide, dibutylamide, butylmethylamide, alkyl ester of N-acyl methioninates, and alkyl N-acetyl methioninate amides of 2-hydroxy-4-(methylthio)butanoic acid.

98. The feed ration of claim 95, wherein the α-amino acid analog is selected from the group consisting of methyl, ethyl, n-propyl, isopropyl, butyl, n-butyl, sec-butyl, isobutyl, tertiary butyl, pentyl, n-pentyl, isopentyl, hexyl, and isohexyl ethers of 2-hydroxy-4-(methylthio)butanoic acid.

99. The feed ration of claim 95, wherein the ester group of the ester/ether of 2-hydroxy-4-(methylthio)butanoic acid is selected from the group consisting of methyl, ethyl, n-propyl, isopropyl, n-butyl, sec-butyl, isobutyl, tertiary butyl, n-pentyl, isopentyl, n-hexyl, and isohexyl esters and the ether group of the ester/ether of 2-hydroxy-4-(methylthio)butanoic acid is selected from the group consisting of methyl, ethyl, n-propyl, isopropyl, n-butyl, sec-butyl, isobutyl, tertiary butyl, n-pentyl, isopentyl, n-hexyl, and isohexyl ethers.

100. The feed ration of claim 95, wherein the α-amino acid analog is selected from the group consisting of zinc, copper, cobalt, manganese, calcium, iron, or magnesium chelates of 2-hydroxy-4-(methylthio)butanoic acid.

101. The feed ration of claim 95, wherein the α-amino acid analog comprises 2-hydroxy-4-(methylthio)butanoic acid.

102. The feed ration of claim 95, wherein the α-amino acid analog comprises the isopropyl ester of 2-hydroxy-4-(methylthio)butanoic acid.

103. The feed ration of claim 95, wherein the α-amino acid analog is selected from the group consisting of zinc, copper, cobalt, manganese, calcium, iron, or magnesium chelates of 2-hydroxy-4-(methylthio)butanoic acid.

104. The feed ration of claim 95, wherein the metal chelate of 2-hydroxy-4-(methylthio)butanoic acid comprises the zinc chelate.

105. The feed ration of claim 104, wherein the molar ratio of 2-hydroxy-4-(methylthio)butanoic acid to zinc is between about 1.5:1 to about 2.5:1.

106. The feed ration of claim 95, wherein the anion salt of 2-hydroxy-4-(methylthio)butanoic acid is selected from the group consisting of zinc, copper, cobalt, manganese, calcium, iron, or magnesium anion salts.

107. The feed ration of claim 106, wherein the anion salt of 2-hydroxy-4-(methylthio)butanoic acid comprises the zinc anion salt.

108. The feed ration of claim 107, wherein the ratio of 2-hydroxy-4-(methylthio)butanoic acid to zinc is between about 0.5:1 to about 1.5:1.

109. The feed ration of claim 95, wherein the α-amino acid analog is selected from the group consisting of ammonium, magnesium, calcium, lithium, sodium, potassium, and zinc salts of methionine.

110. The feed ration of claim 95, wherein the α-amino acid analog is selected from the group consisting of methyl, ethyl, n-propyl, isopropyl, n-butyl, sec-butyl, isobutyl, tertiary butyl, n-pentyl, isopentyl, -hexyl, and isohexyl esters of methionine.

111. The feed ration of claim 95, wherein the α-amino acid analog is selected from the group consisting of methylamide, dimethylamide, ethylmethylamide, butylamide, dibutylamide, butylmethylamide, alkyl ester of N-acyl methioninates, and alkyl N-acetyl methioninates of methionine.

112. The feed ration of claim 95, wherein the α-amino acid analog is selected from the group consisting of methyl, ethyl, n-propyl, isopropyl, n-butyl, sec-butyl, isobutyl, tertiary butyl, n-pentyl, isopentyl, -hexyl and isohexyl ethers of methionine.

113. The feed ration of claim 95, wherein the α-amino acid analog is selected from the group consisting of zinc, copper, cobalt, manganese, calcium, iron, or magnesium chelates of methionine.

114. The feed ration of claim 95, wherein the metal chelate of methioinine comprises the zinc chelate of methionine.

115. The feed ration of claim 114, wherein the molar ratio of methionine to zinc is between about 1.5:1 to about 2.5:1.

116. The feed ration of claim 95, wherein the α-amino acid analog is selected from the group consisting of zinc, copper, cobalt, manganese, calcium, iron, or magnesium anion salts of methionine.

117. The feed ration of claim 116, wherein the anion salt of methionine comprises the zinc anion salt of methionine.

118. The feed ration of claim 117, wherein the ratio of methionine to zinc is between about 0.5:1 to about 1.5:1.

119. The feed ration of claim 95, wherein the analog of methionine is selected from the group consisting of the isopropyl ester, and tertiary butyl ester of methionine.

120. The feed ration of claim 95, wherein the α-amino acid analog is incorporated in the feed ration in a quantity to provide the ruminant about 0.1 and 50 grams of α-amino acid analog per day.

121. The feed ration of claim 95, wherein the α-amino acid analog is incorporated in the feed ration in a quantity to provide the ruminant about 1 and 25 grams of α-amino acid analog per day.

122. The feed ration of claim 95, wherein the α-amino acid analog is incorporated in the feed ration in a quantity to provide the ruminant about 5 and 20 grams of α-amino acid analog per day.

123. The feed ration of claim 95, wherein between about 4 and about 30 pounds of the feed ration comprises between about 0.1 to about 50 grams of α-amino acid analog.

124. The feed ration of claim 95, wherein between about 4 and about 30 pounds of the feed ration comprises between about 1 to about 25 grams of α-amino acid analog.

125. The feed ration of claim 95, wherein between about 4 and about 30 pounds of the feed ration comprises between about 5 to about 20 grams of α-amino acid analog.

126. The feed ration of claim 95, wherein between about 4 and about 30 pounds of the feed ration comprises between about 0.1 to about 50 grams of α-amino acid analog.

127. The feed ration of claim 95, wherein between about 4 and about 12 pounds of the feed ration comprises between about 1 to about 25 grams of α-amino acid analog.

128. The feed ration of claim 95, wherein between about 4 and about 12 pounds of the feed ration comprises between about 5 to about 20 grams of α-amino acid analog.

129. The feed ration of claim 95, wherein between about 4 and about 30 pounds of the feed ration comprises between about 10 to about 18 grams of α-amino acid analog.

130. The feed ration of claim 95, wherein the formulated feed ration is a daily feed ration comprising between about 4 and about 12 pounds of feed ingredients and between about 0.1 to about 50 grams of α-amino acid analog.

131. The feed ration of claim 95, wherein between about 8 and about 24 pounds of the feed ration comprises between about 0.1 to about 50 grams of α-amino acid analog.

132. The feed ration of claim 95, wherein between about 8 and about 24 pounds of the feed ration comprises between about 1 to about 25 grams of α-amino acid analog.

133. The feed ration of claim 95, wherein between about 8 and about 24 pounds of the feed ration comprises between about 5 to about 20 grams of α-amino acid analog.

134. The feed ration of claim 95, further comprising a lysine analog.

135. The feed ration of claim 134, wherein the lysine analog is selected from a the group consisting of lysine salts, amides, esters, ethers and oligomers.

136. The feed ration of claim 135, wherein the amount of lysine analog and the methionine analog is incorporated in the feed ration such that the ratio of the lysine analog to methionine analog available for absorption by the ruminant is between about 1:1 to about 5:1.

137. The feed ration of claim 95, comprising at least 60% forage ingredients.

138. The feed ration of claim 95, comprising at least 70% forage ingredients.

139. The feed ration of claim 95, further comprising molasses.

140. The feed ration of claim 95, wherein an amount of feed ration that is capable of being consumed by a ruminant in a day comprises at least 0.5 pounds of molasses.

141. The feed ration of claim 95, wherein an amount of feed ration that is capable of being consumed by a ruminant in a day comprises between about 1 to about 7 pounds of molasses.

142. The feed ration of claim 95, wherein the ratio of forage to molasses feed ingredients in the ration is between about 20:1 to about 1.5:1.

143. The feed ration of claim 95, wherein the feed ration satisfies the nutritional requirements for methionine.

144. The feed ration of claim 95, wherein the feed ration does not satisfy the nutritional requirements for methionine.

145. A feed ration for growing ruminants comprising:
one or more feed ingredients and the calcium salt of 2-hydroxy-4-(methylthio)butanoic acid;
wherein the feed ration comprises at least 80% forage; and
the feed ration being formulated such that an amount of feed ration that is capable of being consumed by a growing ruminant in a day satisfies the ruminant's daily nutritional requirements, and exceeds daily maintenance energy requirements of a growing ruminant; provided that such assortment and composition may not necessarily satisfy the growing ruminant's methionine requirements.

146. The feed ration of claim 145, wherein the calcium salt of 2-hydroxy-4-(methylthio)butanoic acid is incorporated in the feed ration in a quantity to provide the ruminant about 0.1 and 50 grams of α-amino acid analog per day.

147. The feed ration of claim 145, wherein the formulated feed ration is a daily feed ration comprising between about 4 and about 30 pounds of feed ingredients and between about 0.1 to about 50 grams of calcium salt of 2-hydroxy-4-(methylthio)butanoic acid.

148. The feed ration of claim 145, further comprising molasses.

149. The method of claim 145, wherein an amount of feed ration that is capable of being consumed by a ruminant in a day comprises at least 0.5 pounds of molasses.

150. The feed ration of claim 145, wherein the feed ration satisfies the nutritional requirements for methionine.

151. The feed ration of claim 145, wherein the feed ration does not satisfy the nutritional requirements for methionine.
